SEQUENCE LISTING (17 sequences)

SEQ ID NO: 1 - DNA, 888 bp, Artificial Sequence
  Region of plasmid pPCR-Script57264, comprising 57264 open reading frame
gggcgaattgggtaccatggtctagaatggcggaagcgggcattaccggcacctggtataaccagctgggcagcacctttattgtgaccgcgggtgcggatggtgcgctgaccggcacctatgaaagcgcggtgggcaacgcggaaagccgttatgtgctgaccggccgttatgatagcgcaccggcgaccgatggtagcggcaccgcgctgggttggaccgtggcgtggaaaaacaactatcgtaacgcgcatagcgcgaccacctggagcggccagtatgtgggtggtgcggaagcgcgtattaacacccagtggctgctgaccagcggcaccaccgaagcgaacgcgtggaaaagcaccctggtgggccatgatacctttaccaaagtgaaaccgagcgcggcgtctggtggtggtggtagcggcggtggcggcagcggtggcggcggtagcggatccttaagcggccgcatgcataccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcctaagaattctcgagctccagcttttgttccc

SEQ ID NO: 2 - protein, 277 aa, Artificial Sequence
  Region of plasmid pPCR-Script57264, comprising 57264 open reading frame
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASGGGGSGGGGSGGGGSGSLSGRMHTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQA

SEQ ID NO: 3 - DNA, 960 bp, Artificial Sequence
  Map of region of plasmid pDUET57264, comprising 57264 open reading frame
ggggaattgtgagcggataacaattcccctctagaaataattttgtttaactttaagaaggagatataccatggcggaagcgggcattaccggcacctggtataaccagctgggcagcacctttattgtgaccgcgggtgcggatggtgcgctgaccggcacctatgaaagcgcggtgggcaacgcggaaagccgttatgtgctgaccggccgttatgatagcgcaccggcgaccgatggtagcggcaccgcgctgggttggaccgtggcgtggaaaaacaactatcgtaacgcgcatagcgcgaccacctggagcggccagtatgtgggtggtgcggaagcgcgtattaacacccagtggctgctgaccagcggcaccaccgaagcgaacgcgtggaaaagcaccctggtgggccatgatacctttaccaaagtgaaaccgagcgcggcgtctggtggtggtggtagcggcggtggcggcagcggtggcggcggtagcggatccttaagcggccgcatgcataccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcctaagaattcgagctcggcgcgcctgcaggtcgacaagcttgcggccgcataatgcttaa

SEQ ID NO: 4 - protein, 277 aa, Artificial Sequence; identical to SEQ ID NO: 2
  Map of region of plasmid pDUET57264, comprising 57264 open reading frame

SEQ ID NO: 5 - DNA, 600 bp, Artificial Sequence
  Map of region of plasmid pDUETNLCPA, comprising NLCPA open reading frame
ggggaattgtgagcggataacaattcccctctagaaataattttgtttaactttaagaaggagatataccatggtaccaagcttaggtggtggtggtagcggcggtggcggcagcggtggcggcggtagcggatccttaagcggccgcatgcataccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcctaagaattcgagctcggcgcgcctgcaggtcgagtctggtaaagaaaccgctgctgcgaaatttgaac

SEQ ID NO: 6 - protein, 154 aa, Artificial Sequence
  Map of region of plasmid pDUETNLCPA, comprising NLCPA open reading frame
MVPSLGGGGSGGGGSGGGGSGSLSGRMHTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQA

SEQ ID NO: 7 - DNA, 1140 bp, Artificial Sequence
  Map of region of plasmid pDUETNPANLCP40, comprising NPANLCP40 open reading frame
ggggaattgtgagcggataacaattcccctctagaaataattttgtttaactttaagaaggagatataccatggttaccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcaagcttaggtggtggtggtagcggcggtggcggcagcggtggcggcggtagcggatccgtgtttccagccacgcgaacgtatgcctgttatgtcgacggcaaggttcacggcaatggcggagacttgaacatgatcaaccctgcgtgtcttgacgccttggcgatctcgggcaactatcaattctggaactggttcggaaatctgatcagtaatgccggaggacgccatagggaaatcattcctgacggcaaactgtgcggaccaacggccagttttgatggtatgaaccaggcgcgtacagactggtggacgactcgtctgcagccgggcgcaacgattacggtgcgagtcaacgcatgggcgccgcatcccggcacgtggtatttgtatgtaacccgggacggatgggacccgacgcaaccattgaaatggtcggatctgggaccgacgcccttcagccaggtgactaatccgccgatcaactcgagcggaccggacggggccgagtacagctggcaggtacagctgccgaacaagcaaggtcgacacatcatttatatgatatggcagagatctgatagtccagaggcattctacaactgttcggatgtttatttcggatcggggccgattgcttatgaatttggtgacccgcgggaaggcggataagaattcgagctcggcgc

SEQ ID NO: 8 - protein, 350 aa, Artificial Sequence
  Map of region of plasmid pDUETNPANLCP40, comprising NPANLCP40 open reading frame
MVTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQASLGGGGSGGGGSGGGGSGSVFPATRTYACYVDGKVHGNGGDLNMINPACLDALAISGNYQFWNWFGNLISNAGGRHREIIPDGKLCGPTASFDGMNQARTDWWTTRLQPGATITVRVNAWAPHPGTWYLYVTRDGWDPTQPLKWSDLGPTPFSQVTNPPINSSGPDGAEYSWQVQLPNKQGRHIIYMIWQRSDSPEAFYNCSDVYFGSGPIAYEFGDPREGG

SEQ ID NO: 9 - DNA, 1200 bp, Artificial Sequence
  Map of region of plasmid pDUETNP40NLCPA, comprising the NP40NLCPA open reading frame
ggggaattgtgagcggataacaattcccctctagaaataattttgtttaactttaagaaggagatataccatggtgtttccagccacgcgaacgtatgcctgttatgtcgacggcaaggttcacggcaatggcggagacttgaacatgatcaaccctgcgtgtcttgacgccttggcgatctcgggcaactatcaattctggaactggttcggaaatctgatcagtaatgccggaggacgccatagggaaatcattcctgacggcaaactgtgcggaccaacggccagttttgatggtatgaaccaggcgcgtacagactggtggacgactcgtctgcagccgggcgcaacgattacggtgcgagtcaacgcatgggcgccgcatcccggcacgtggtatttgtatgtaacccggaacggatgggacccgacgcaattattgaaatggtcggatctggaaccgacgcccttcagccaggtgactaatccgccgatcaactcgagcggaccggacggggccgagtacagctggcaggtacagctgccgaacaagcaaggtcgacacatcatttatatgatgtggcagagatctgatagtccagaggcattctacaactgttcggatgtttatttcggatcggggccgattgcttatgaatttggtgacccgcgagaaggcggaagcttaggtggtggtggtagcggcggtggcggcagcggtggcggcggtagcggatccttaagcggccgcatgcataccccggcagcgaatgcggcgcagcatgatgaagcggtggataacaaattcaacaaagaacagcagaacgcgttttatgaaattctgcatctgccgaacctgaacgaagaacagcgtaatgcgtttattcagagcctgaaagatgatccgagccagagcgcgaacctgctggcggaagcgaaaaaactgaacgatgcgcaggcgccgaaagtggataataaatttaataaagaacagcagaatgccttctacgaaatcctgcacctgccgaatctgaatgaagaacagcgcaacgccttcatccagagcctgaaagatgatccgagccagagcgccaatctgctggccgaagccaaaaaactgaatgatgcccaggcctaagaattcgagctcggcgcgcctgcaggtcgagtctggtaaagaaaccgctgctgcgaaatttg

SEQ ID NO: 10 - protein, 355 aa, Artificial Sequence
  Map of region of plasmid pDUETNP40NLCPA, comprising the NP40NLCPA open reading frame
MVFPATRTYACYVDGKVHGNGGDLNMINPACLDALAISGNYQFWNWFGNLISNAGGRHREIIPDGKLCGPTASFDGMNQARTDWWTTRLQPGATITVRVNAWAPHPGTWYLYVTRNGWDPTQLLKWSDLEPTPFSQVTNPPINSSGPDGAEYSWQVQLPNKQGRHIIYMMWQRSDSPEAFYNCSDVYFGSGPIAYEFGDPREGGSLGGGGSGGGGSGGGGSGSLSGRMHTPAANAAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQA

SEQ ID NO: 11 - protein, 111 aa, Artificial Sequence
  Protein sequence of a fusion protein with a synthetic particle forming domain and a synthetic affinity peptide domain
MIIIIIIIIIIVVVVVVVVVVLLLLLLLLLLFFCCIILLAALLVVAAAAAAAGGGGSGGGGSGGGGSGGGGSGSLSGRMHKGGGEPDLFECLHQPYWAIRDCFWFDGTGEA

SEQ ID NO: 12 - DNA, 780 bp, Artificial Sequence
  DNA sequence encoding the protein sequence of a fusion protein with a synthetic particle forming domain and a synthetic affinity peptide domain
gggcgaattgggtaccatggtctagaatgatcattattatcattatcatcatcattattgcccgcttaacccatggtaccagatcttactagtaataatagtaatagtagtagtaataactggtggtggttgtggttgttgtggtcgttctgctgctgctgctgctgctgctgctgctgtaccaccaccaacaccaacaacaccagcaagacgacgacgacgacgacgacgacgacgacattttctgctgcattattctgctggcggcgctgctggttgttgcagcggcagcagcagcggaaaagacgacgtaataagacgaccgccgcgacgaccaacaacgtcgccgtcgtcgtcgcccaggtggtggcggttctggcggtggtggcagcggtggcggtggttcaggtggtggtggttgtccaccaccgccaagaccgccaccaccgtcgccaccgccaccaagtccaccaccaccaactggatccttaagcggccgcatgcataaaggcggcggtgaaccggacctgtttgaatgccgacctaggaattcgccggcgtacgtatttccgccgccacttggcctggacaaacttacggtgcatcagccgtattgggcgattcgtgattgcttttggtttgatggcaccggcgaagcgtacgtagtcggcataacccgctaagcactaacgaaaaccaaactaccgtggccgcttcgcaaagaattctcgagctccagcttttgttcccttcttaagagctcgaggtcgaaaacaaggg

SEQ ID NO: 13 - DNA, 567 bp, Artificial Sequence
  Map of CD4 domains 1 and 2 (CD4d12) PCR product
aaaaccatggctaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcaagctttttt

SEQ ID NO: 14 - protein, 185 aa, Artificial Sequence
  Map of CD4 domains 1 and 2 (CD4d12) PCR product
MAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKAS

SEQ ID NO: 15 - protein, 400 aa, Artificial Sequence [sequence omitted]

SEQ ID NO: 16 - DNA, 31 bp, Artificial Sequence
  Primer
aaaaccatggctaagaaagtggtgctgggca

SEQ ID NO: 17 - DNA, 29 bp, Artificial Sequence
  Primer
aaaaaagcttgccttctggaaagctagca

(19) United States
(12) Patent Application Publication
(10) Pub. No.: US 2012/0009624 A1
(43) Pub. Date: Jan. 12, 2012

(54) PROTEIN PARTICLES

(76) Inventors: Jens Sommer-Knudsen, New south Wales (AU); Moreland D. Gibbs, New south Wales (AU)

(21) Appl. No.: 12/517,325

(22) PCT Filed: Dec. 3, 2007
(86) PCT No.: PCT/AU2007/001863

§ 371 Date: Sep. 22, 2011

(30) Foreign Application Priority Data
Dec. 4, 2006 (AU) 2006906777

(51) Int. Cl. C07K 19/00; C12P 21/00; C07K 1/107; C12N 7/00; B01D 15/00; C12N 9/00; C07K 1/14; C07H 1/06; C12N 1/00; C12N 9/96; C12N 7/02

(52) U.S. Cl. 435/69.7; 530/350; 530/367; 530/387.3; 530/396; 435/188; 530/399; 530/409; 530/395; 530/358; 435/235.1; 435/239; 435/183; 530/387.1; 536/25.4; 530/344; 536/123.1; 536/127; 435/243; 210/660

(57) ABSTRACT

The invention relates to a protein particle comprising chimeric protein having an aggregating part capable of forming or aggregating into a substantially insoluble protein particle when expressed by a cell; and a functional part capable of binding to, or being bound by, a target compound. Affinity matrixes comprising the protein particle are also provided.

### TECHNICAL FIELD

The present invention relates to proteinaceous structures/particles of chimeric protein. The chimeric protein has one part capable of forming or aggregating into an insoluble part and at least one part capable of performing a biological or chemical function.

### BACKGROUND OF THE INVENTION

Insoluble particles with proteins or peptides capable of performing a biological or chemical function displayed on the surface or internal porous areas are typically prepared by first forming a particle of a suitable material such as an organic or inorganic polymer, metal, or ceramic material, attaching chemically active groups to this material and then in turn immobilising the desired peptide or protein in a purified form to the particle through reaction with said chemically active groups. The production of such particles is often cumbersome and expensive as it typically involves several steps, including formation of the particles themselves, activation of these particles, synthesis or purification of the desired peptide or protein as well as the step of immobilising the purified peptide or protein to the particle.

The present inventor has found that useful protein particles can be made that have applications which include separation of compounds.

### SUMMARY OF THE INVENTION

In a first aspect, the present invention provides a protein particle comprising:

chimeric protein having an aggregating part capable of forming or aggregating into a substantially insoluble protein particle when expressed by a cell; and

a functional part capable of binding to, or being bound by, a target compound.

The aggregating part of the protein can be obtained or derived from any suitable protein that can form aggregates such as a synthetic peptide, naturally occurring peptide, or mutant peptide capable of forming aggregates in a cell. In one preferred from, the aggregating part may be P40. It will be appreciated that the aggregating part may be a known protein or part thereof or an artificially formed protein or peptide that, when expressed in a cell, forms aggregates.

The protein particle may comprise two or more functional parts capable of binding to, or being bound by, a two or more target compounds.

In a second aspect, the present invention provides a nucleic acid molecule encoding a protein particle comprising:

a nucleic acid molecule encoding a chimeric protein having an aggregating protein part and a functional protein part capable of binding to, or being bound by, a target compound;

wherein when the nucleic acid molecule is expressed in a cell a protein particle capable of binding to, or being bound by, a target compound is formed.

In a third aspect, the present invention provides a method of forming a protein particle capable of binding to, or being bound by, a target compound comprising:

providing a nucleic acid molecule according to the second aspect of the present invention to a cell;

allowing the cell to express the nucleic acid molecule to form an insoluble protein particle; and

recovering the insoluble protein particle.

The nucleic acid molecule can be provided in any suitable construct such as vector, plasmid, virus, or any other suitable expression means.

In a fourth aspect, the present invention provides a protein affinity matrix for binding or separating at least one target component from a mixture comprising:

a protein particle comprising an aggregating part capable of forming or aggregating into an insoluble protein particle when expressed by a cell; and

a functional part capable of binding to, or being bound by, a target compound.

The affinity matrix may comprise a plurality of protein particles.

The protein particle may contain one or more different functional parts capable of binding one or more different target components.

The functional part may comprise protein A, protein G, protein L, an antibody binding domain, a single chain antibody, avidin, streptavidin, an enzyme, an inhibitor, an antigenic determinant, an epitope, a binding site, a lectin, a cellulose binding protein, a polyhistidine, an oligohistidine, a receptor, a hormone, a signalling molecule, an affinity peptide or protein, a polypeptide with specific or group specific binding capabilities, or any combination thereof.

The protein particle is preferably produced by recombinant DNA technology. The protein forming the particle is a chimeric protein made from two different proteins or parts of proteins from the same or different species.

In a fifth aspect, the present invention provides a method for separating at least one target component from a mixture comprising:

providing a sample containing a target component to an affinity matrix according to the fourth aspect of the present invention; and

allowing a target component in the sample to bind to the functional part of the matrix.

Preferably, the method further comprises:

recovering the target component from the matrix.

The method according to the present invention can be used to enrich at least one desired component within a mixture by separating at least one undesired target component from the mixture.

When a mixture is contacted with the affinity matrix the target component selectively binds, or is selectively bound by, the functional part of the protein. The mixture may comprise any suspension, dispersion, solution or combination thereof of any biological extracts or derivatives thereof. For example, the mixture may include blood, blood plasma, blood serum, blood derived precipitates or supernatants, animal extracts or secretions, milk, colostrum, whey or any other milk derived product or fraction thereof, fermentation broths, liquids or fractions thereof, cell lysates, cell culture supernatants, cell extracts, cell suspensions, viral cultures or lysates, plant extracts or fractions thereof.

The target component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, microorganism or protista.

The target component may be a desired target component or an undesired target component. The undesired target component may be a contaminant.

The target component may be recovered from the affinity matrix to which the target component is bound. The recovery of the target component may be via at least one elution step wherein the binding of the target component to the protein is weakened, disrupted, broken or competitively substituted.

The affinity matrix according to the present invention may be used to obtain a desired component or an undesired component sample.

The desired component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, microorganism or protista.

The undesired target component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, microorganism or protista.

In a sixth aspect, the present invention provides use of the affinity matrix according to the fourth aspect of the present invention to separate or enrich at least one target component.

In a seventh aspect, the present invention provides a kit for affinity separation comprising:

an affinity matrix according to the fourth aspect of the present invention; and

diluent and/or eluent for carrying out an affinity separation using the matrix.

In a preferred form, the kit further contains instructions to carry out an affinity separation.

An advantage of the present invention is the ability to produce recombinantly and recover the particles when made by a cell. For example, the particles can be recovered by centrifugation, sedimentation or filtration.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred embodiments will be described with reference to the following drawings and examples.

### BRIEF DESCRIPTION OF THE DRAWINGS

FIG. 1 shows depictions of possible protein particle forming/functional domain combinations. A. Shows a linear depiction of a hypothetical recombinant protein with an N-terminal functional domain, joined by a linker peptide to a C-terminal protein particle forming domain (PPF-domain). B. Shows a linear depiction of a hypothetical recombinant protein with a C-terminal functional domain, joined by a linker peptide to an N-terminal protein particle forming domain (PPF-domain). C. Shows linear depictions of various domain combinations possible when two functional domains are incorporated into the recombinant protein. D. Shows a linear depiction of a two hypothetical recombinant proteins with different C-terminal functional domains, both joined by a linker peptide to an N-terminal protein particle forming domain (IB-domain). These two peptides would be co-expressed to form protein particles formed from two proteins with differing functional domain.

FIG. 2. Depictions of the cassette regions, and PCR products. A. A linear depiction of the cassette region of the of the plasmid pPCR-Script:57264. The relative binding positions of the oligonucleotide 57264F and M13R primers used to amplify the 57264 cassette for transfer into pDuet-1 are shown as arrows, and are drawn to depict 5′-3′ binding orientation. B. A linear depiction of the cassette region of the of the plasmid pDuet:57264. C. A linear depiction of the cassette region of the of the plasmid pDuet:57264SX. D. A linear depiction of the new linker (NL) PCR product incorporated into the plasmid pDuet:57264SX to create pDuet:NLCPA. Oligonucleotides are depicted as for 2A. E. A linear depiction of the cassette region of the of the plasmid pDuet: NLCPA.

FIG. 3. A. A linear depiction of the P40 region of the of the plasmid pSUN30. B. A linear depiction of the PCR products amplified using the oligonucleotide primer combinations M13RMG+P40BAMR and P40BAMF+NP40R. Relative binding positions of oligonucleotides are shown as arrows, and are drawn to depict 5′-3′ binding orientation. C. A linear depiction of the NP40 domain, now lacking a BamHI site, generated by overlap extension PCR of the two products depicted in 3B. D. A linear depiction of the plasmid pDuet:NP40NLCPA.

FIG. 4. A. A linear depiction of the NP40 region of the of the plasmid pDuet:NP40NLCPA used as template for PCR using the primers CP40F 3+CP40R2 to amplify the CP40 fragment. Relative binding positions of oligonucleotides are shown as arrows, and are drawn to depict 5′-3′ binding orientation. B. A linear depiction of the CP40 PCR product. C. A linear depiction of the cassette region of the plasmid pDuet:NLCP40. D. A linear depiction of the Protein A region of the of the plasmid pDuet:NLCPA used as template for PCR using the primers NPAF+NPAR to amplify the NPA fragment. Relative binding positions of oligonucleotides are shown as arrows, and are drawn to depict 5′-3′ binding orientation. E. A linear depiction of the NPA PCR product. F. A linear depiction of the cassette region of the plasmid pDuet:NPANLCP40.

FIG. 5. SDS-PAGE analysis of soluble and insoluble protein fractions extracted from BL21-Tune:pDuet:NP40NLCPA. Lane 1, 20 μl insoluble fraction after second wash; Lane 2, 20 μl soluble fraction; Lane 3, 20 μl first wash soluble fraction; Lane 4, 20 μl second wash soluble fraction; Lane 5, Prestained Protein molecular weight marker (Fermentas).

FIG. 6. SDS-PAGE analysis of soluble and insoluble protein fractions extracted from BL21-Tuner:pDuet:NPANLCP40. Lane 1, 20 μl insoluble fraction after second wash; Lane 2, 20 μl soluble fraction; Lane 3, 20 μl first wash soluble fraction; Lane 4, 20 μl second wash soluble fraction; Lane 5, Prestained Protein molecular weight marker (Fermentas).

FIG. 7. SDS-PAGE analysis of Immunoglobulins purified from human serum.

### M: Marker; 5 μl (Invitrogen);

S: Human serum (diluted 1+4); 5 μl

### 1: Elute of NPANLCP40; 5 μl

2: Elute of NP40NLCPA (diluted 1+1); 5 μl
3: Elute of negative control; 5 μl

### 4: Elute of NPANLCP40; 10 μl

5: Elute of NP40NLCPA (diluted 1+1); 10 μl
6: Elute of negative control; 10 μl

FIG. 8 Map of region of plasmid pPCR-Script:57264, comprising 57264 open reading frame. DNA sequence is (SEQ ID NO: 1) and translated peptide sequence is (SEQ ID NO: 2).

FIG. 9 shows map of region of plasmid pDUET57264, comprising 57264 open reading frame. DNA sequence is (SEQ ID NO: 3) and translated peptide sequence is (SEQ ID NO: 4).

FIG. 10 shows map of region of plasmid pDUETNLCPA, comprising NLCPA open reading frame: DNA sequence is (SEQ ID NO: 5) and translated peptide sequence is (SEQ ID NO: 6).

FIG. 11 shows map of region of plasmid pDUETNPANLCP40, comprising NPANLCP40 open reading frame. DNA sequence is (SEQ ID NO: 7) and translated peptide sequence is (SEQ ID NO: 8).

FIG. 12 shows map of region of plasmid pDUETNP40NLCPA, comprising the NP40NLCPA open reading frame. DNA sequence is (SEQ ID NO: 9) and translated peptide sequence is (SEQ ID NO: 10).

FIG. 13 shows protein sequence of a fusion protein with a synthetic particle forming domain and a synthetic affinity peptide domain. Amino acids 1 to 52 constitute a synthetic particle forming domain, amino acids 53 to 74 constitute a linker peptide and amino acid 75 to 111 constitute a synthetic affinity peptide domain. Peptide sequence is (SEQ ID NO: 11).

FIG. 14 shows DNA sequence encoding the protein sequence shown in FIG. 13. DNA sequence is (SEQ ID NO: 12).

FIG. 15 shows Map of CD4 domains 1 and 2 (CD4d12) PCR product. DNA sequence is (SEQ ID NO: 13) and translated peptide sequence is (SEQ ID NO: 14).

FIG. 16 shows predicted translated gene product from pDuet:NCD4NLP40. Peptide sequence is (SEQ ID NO: 15).

FIG. 17 shows depictions of PCR products and cassette regions. A. A linear depiction of CD4 domains 1 and 2 (CD4d12) PCR product. The PCR product was amplified, using the primers CD4F and CD4R, from a plasmid containing a copy of human CD4 cDNA. The relative binding positions of the CD4F and CD4R primers used to amplify the CD4d12 cassette for transfer into pDuet:NLCP40 are shown as arrows, and are drawn to depict 5′-3′ binding orientation. B. A linear depiction of the cassette region of the of the plasmid pDuet:NLCP40. C. A linear depiction of the cassette region of the of the plasmid pDuet:57264SX.

FIG. 18 shows SDS-PAGE analysis of NCD4NLCP40 particles. Soluble and insoluble fractions of E. coli lysates were prepared using BPER (Pierce). Arrow indicates NCD4NLCP40 protein.

### MODE(S) FOR CARRYING OUT THE INVENTION

### Definitions

The term “particle” as used herein refers to a substantially insoluble entity consisting of a protein. These entities may be spherical, ellipsoidal, in string form, in sheets, discs or any other shape. The particles may be of any size between 1 nm and 100 μm.

The term “polypeptide” as used herein means a polymer made up of amino acids linked together by peptide bonds, and includes fragments or analogues thereof. The terms “polypeptide” and “protein” are used interchangeably herein, although for the purposes of the present invention a “polypeptide” may constitute a portion of a full length protein or a complete full length protein.

The term “nucleic acid” as used herein refers to a single- or double-stranded polymer of deoxyribonucleotide, ribonucleotide bases or known analogues of natural nucleotides, or mixtures thereof. The term includes reference to a specified sequence as well as to a sequence complimentary thereto, unless otherwise indicated. The terms “nucleic acid” and “polynucleotide” are used herein interchangeably.

The term “variant” as used herein refers to substantially similar sequences. Generally, polypeptide sequence variant possesses qualitative biological activity in common. Further, these polypeptide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides of the invention. A homologue is typically a polypeptide from a different species but sharing substantially the same biological function or activity as the corresponding polypeptide disclosed herein. Variant therefore can refer to a polypeptide which is produced from the nucleic acid encoding a polypeptide, but differs from the wild type polypeptide in that it is processed differently such that it has an altered amino acid sequence. For example a variant may be produced by an alternative splicing pattern of the primary RNA transcript to that which produces a wild type polypeptide.

The term “fragment” refers to a polypeptide molecule that encodes a constituent or is a constituent of a polypeptide of the invention or variant thereof. Typically the fragment possesses qualitative biological activity in common with the polypeptide of which it is a constituent. The term “fragment” therefore refers to a polypeptide molecule that is a constituent of a full-length polypeptide and possesses at least some qualitative biological activity in common with the full-length polypeptide. The fragment may be derived from the full-length polypeptide or be expressed as is from a suitable organisms containing nucleic acid encoding a fragment of the full-length polypeptide

The term “substantially” as used herein means the majority but not necessarily all, and thus in relation to a modified polypeptide “substantially” lacking a component region of a corresponding wild-type polypeptide, the modified polypeptide may retain a portion of that component region. For example, a modified polypeptide “substantially” lacking a component region of a corresponding wild-type polypeptide may retain approximately 50 percent or less of the sequence of the component region, although typically the component region is rendered structurally and/or functionally inactive by virtue of the proportion of the sequences of the region omitted.

The term “affinity separation” as used herein refers to a method of separating, purifying, removing, enriching and/or concentrating a component from a mixture or suspension.

The term “chimeric protein” as used herein means a protein produced by expression of a recombinant nucleic acid encoding a protein having at least two parts, one part capable of forming or aggregating into an insoluble particle and at least a second part capable of a biologically or chemically relevant function. The two parts can come from the same species, from different species or be synthetic.

### Outline

The present invention is predicated on the finding that insoluble particles of peptides or proteins are capable of performing a biological or chemical function can be obtained through expression of chimeric recombinant proteins where one part of the protein is capable of forming an insoluble particle and the other part of the protein is performing the desired biological or chemical function. These self assembling structures/particles can be made by producing a nucleic acid, typically DNA, construct encoding a peptide/protein chain which will form an insoluble particle linked with a sequence encoding at least one protein or peptide capable of biological function or interaction and expressing this DNA construct in a suitable host organism. The self-assembling core may be a peptide/protein known to form inclusion bodies (IB) when expressed in a suitable manner in a suitable host, or it may be a specially designed sequence capable of forming an insoluble particle having the desired characteristics. The two sequences may or may not be interspaced by a sequence encoding a non-hydrophobic “spacing” peptide or protein sequence. The size of the structures/particles would depend on the length of the engineered protein chain an may be in the range of about 1 nm to 5 μm if assembled inside the producing cell and up to several hundred micrometer if assembled outside the cell such as when the protein chains are secreted into the medium surrounding the cells (e.g. by including a nucleotide sequence encoding a secretion signal peptide) or when the structures are assembled in vitro.

The structures may be made up of heterologous protein strands with different protein particle forming sequences and different biologically relevant proteins/peptides such that each of these structures will carry more than one type of biologically relevant molecule on the surface.

The host organism for expressing the protein may be a prokaryotic organism or a eukaryotic organism. The prokaryotic organism may be a bacterium and the eukaryotic organism may be a yeast, a fungus, a protist, a plant, an animal, or cultures of any of combination thereof.

While it is expected that these self assembling protein particles have a wide range of applications, it has been found that these particles can be used for affinity separations, where the part of the protein capable of a desired biological function binds to, or is being bound by, a desired target component.

### Self Assembling Protein Particles

The present invention is based on the surprising and unexpected finding that functional, self assembling proteinaceous particles can be prepared by providing a nucleic acid construct encoding a chimeric protein where one part is capable of forming or aggregating into an insoluble particle and one part capable of a biologically relevant function or interaction while being displayed on the particle, expressing said DNA construct in a suitable host organism and preferably recovering said particles from said host organism.

### Chimeric Proteins

The present invention contemplates production of recombinant chimeric proteins that have been modified to contain at least one part that forms or aggregates into an insoluble particle and at least one part that is capable of a biologically or chemically relevant function. Typically these proteins are created by recombinant DNA technology where nucleotide fragments encoding the desired proteins, peptides or fragments thereof are joined together with or without an interspaced nucleotide fragment encoding a spacer or linker region. One part of the protein may be the protein P40 or any other protein such as Alpha-amylase, human alpha-fetoprotein, Somatotropin, cellulose binding domain from clostridium, or other proteins such as synthetic proteins or peptides, which forms or aggregates into suitable particles when expressed in an appropriate host organism such as Escherichia coli, and at least one other part of the protein may comprise an antibody binding domain such as protein A, protein G, protein L, or a single chain antibody, avidin, streptavidin, an enzyme, an inhibitor, an antigenic determinant, an epitope, a binding site, a lectin, a polyhistidine, an oligohistidine, a receptor, a hormone, a signalling molecule, a polypeptide with specific or group specific binding capabilities, or any combination thereof.

### Affinity Separations

An example of an application of the present invention is based on the finding that these self assembling protein particles can be used for affinity separations thus providing for relatively inexpensive and reliable affinity separations. The particular instance of affinity separation exemplified herein is readily understood and appreciated by persons skilled in the art as representing a general method of affinity separation suitable for the separation, purification, removal, enrichment and/or concentration of any desired or undesired component.

Accordingly, the present invention in a preferred form relates to an affinity matrix separating at least one target component from a mixture. The affinity matrix comprises at least one protein part having at least one part capable of forming an insoluble particle and at least one part able to bind the target component of interest. When a mixture or sample is contacted with the affinity matrix, the target component selectively binds to a part of the protein.

Thus, binding of the target component to the affinity matrix allows the target component to be separated from the mixture. The target molecules can, if desired, be separated from the affinity matrix by elution through methods well known to persons skilled in the art.

### Target Components

The target component may comprise a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a virus, a biological extract, a hormone, a serum protein, a milk protein, a milk-derived product, blood, serum, plasma, a fermentation product a macromolecule or any other molecule or any combination or fraction thereof. The biological extract may be derived from any plant, animal, micro-organism or protista.

The target component may be a desired target component, such as an immunoglobulin from serum. However, the target component may also be undesired, such as a contaminant.

### Binding of Target Component(s) to Affinity Matrix

Target components may be bound to the affinity matrix by conventional methods such as those usually employed in affinity separations. These include: 1) packing the matrix in a column and passing the mixture containing the target component through the packed column; 2) adding the matrix to a vessel such as employed in fluid bed separations followed by passing the mixture through the affinity matrix in a manner that causes the affinity matrix to become fluidised; 3) mixing the affinity matrix with the mixture in a vessel and subsequently separating the affinity matrix containing the target component from the mixture by means of sedimentation, centrifugation, or filtration.

### Recovery of Target Component(s)

The target component may be recovered from the affinity matrix to which the target component is bound, and this recovery may involve at least one elution step. In this regard, the relevant eluant(s) may comprise a solution with compounds imparting high or low pH, high or low salt concentrations or compounds with competitive binding capacity. Such solutions can comprise inorganic or organic acids or salts thereof, chaotropic salts, or compounds with competitive binding capacity. For example, a buffer comprising glycine adjusted to a pH in the range of about 1.5 to 4. Other examples include buffers comprising citric, acetic, succinic, lactic, tartric, formic, propionic, boric or phosphoric acids or salts thereof. The eluant may also comprise a solution of one or more inorganic acids, for example hydrochloric acid, sulphuric acid and nitric acid, or salts thereof such as sodium chloride, potassium chloride, ammonium chloride, sodium sulphate, potassium sulphate or ammonium sulphate. The eluant may also comprise a solution of one or more organic or inorganic basic compounds or salts thereof such as methylamine, piperazine, carbonate, phosphate, borate or ammonium hydroxide. The eluant may also comprise chaotropic compounds such as urea, guanidine, potassium iodide, sodium iodide, thiocyanates, detergents, hydrophobic molecules such as organic solvents, or any other molecule capable of weakening, breaking or disrupting molecular structures or bonds.

The eluant(s) used in the elution step(s) may have a pH in the range of about 1.0 to about 14.0. The eluants may have ionic strengths in the range from about 1×10⁻³ to about 25.

### Kits

The present invention also provides kits for separating, purifying, removing, enriching and/or concentrating a component from a mixture or suspension, wherein the kits facilitate the employment of the systems and methods of the invention. Typically, kits for carrying out a method of affinity separation contain at least a number of the reagents required to carry out the method. Typically, the kits of the invention will comprise one or more containers, containing for example, matrices, wash reagents, and/or other reagents capable of releasing a bound component from a polypeptide or fragment thereof.

In the context of the present invention, a compartmentalised kit includes any kit in which matrices and/or reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept a test sample, a container which contains the affinity matrices used in the assay and containers which contain wash reagents (such as phosphate buffered saline, Tris-buffers, and the like).

Typically, a kit of the present invention will also include instructions for using the kit components to conduct the appropriate methods.

Methods and kits of the present invention find application in any circumstance in which it is desirable to purify any component from any mixture.

### EXAMPLES

### Example 1

### Preparation of a Functional Protein Particle

The following example describes the creation of a two recombinant genes, and the expression of said genes as multi-domain proteins comprising a protein particle forming domain (ppf-domain) and a Protein A domain with affinity for immunoglobulins from a number of mammalian species.

A number of preliminary recombinant DNA manipulations were performed to create the final recombinant genes described in this exemplification.

All enzymatic manipulations of DNA, the polymerase chain reaction, and oligonucleotide designs, described below, were performed essentially according to the accepted art, as described in Sambrook et al (2000, Molecular Cloning: A Laboratory Manual [Third Edition], Cold Spring Harbor Laboratories, NY 1172, USA), and have not been described in detail here. At all appropriate stages, new plasmids constructions were sequenced across any modified regions or newly incorporated regions, to confirm that the DNA sequence was intact, and matched the expected sequence.

### Preliminary Vector Construction

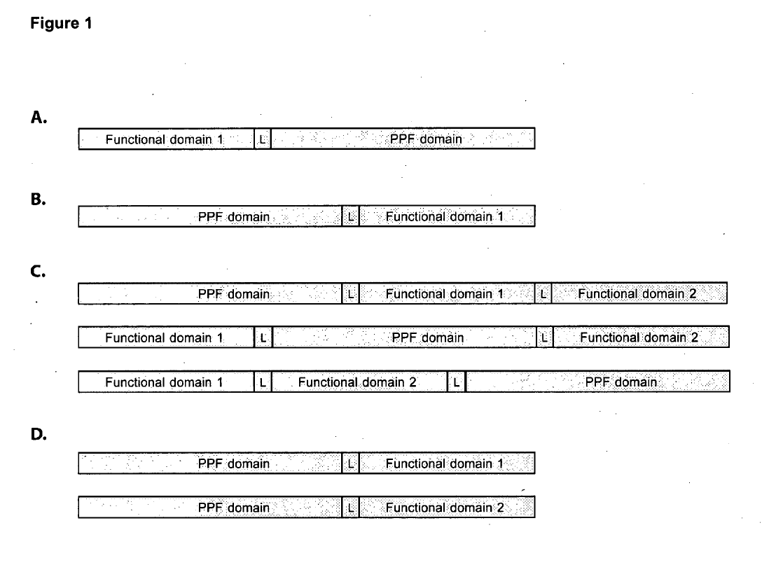

A recombinant gene cassette was designed by the inventor, and synthesized de novo by GeneART (GENEART AG, BioPark Josef-Engert-Str., 11 D-93053, Regensburg, Germany). The gene cassette was supplied as plasmid DNA, referred to herein, as plasmid pPCR-Script:57264, in the general cloning vector pPCR-Script (Stratagene, 11011 N. Torrey Pines Road, La Jolla, Calif. 92037, USA). The pPCR-Script:57264 plasmid incorporated two restriction enzyme sites NcoI and EcoRI flanking the gene cassette (See FIG. 1A., Seq #1). The 57264 cassette was designed to encode a protein consisting of an N-terminal Streptavidin domain, a central glycine rich linker, and a C-terminal Protein A domain (Seq #2).

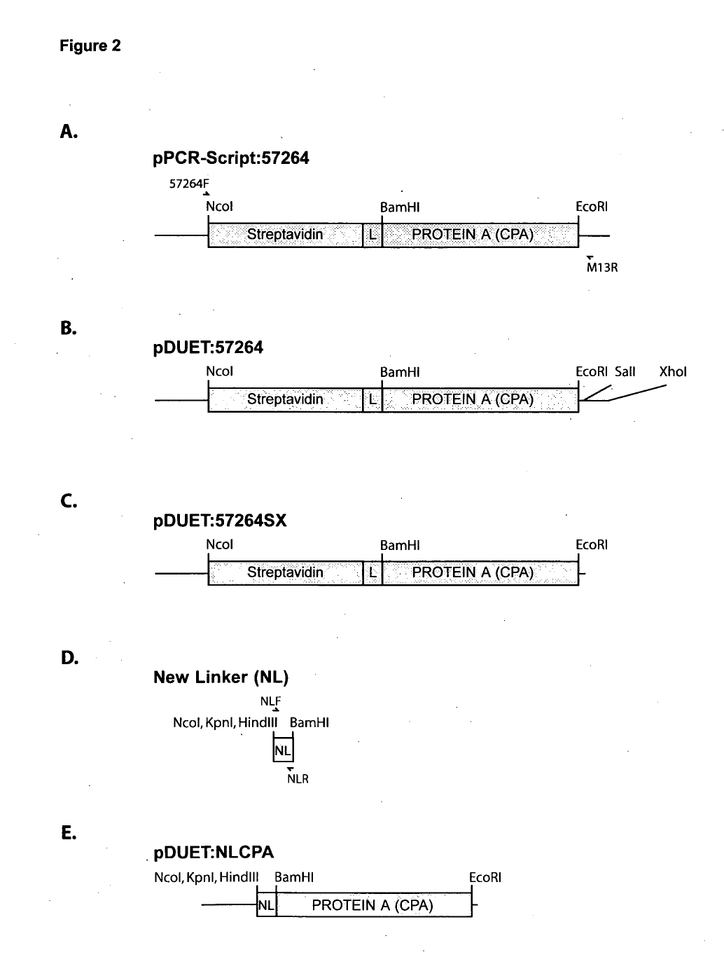

The oligonucleotide primers 57264F and M13R (See Table 1 & FIG. 2A) were used to amplify the 57264 cassette from the pPCR-Script:57264 plasmid using the PCR. The 57264F primer was designed to incorporate the restriction enzyme site NcoI, directly in-frame with the start codon of the 57264 cassette open reading frame (ORF). Incorporation of the restriction site allowed restriction digestion of the PCR product with NcoI and EcoRI, and subsequent directional ligation of the digested PCR product into the same sites in the controlled-expression vector pDuet-1 (Novagen, EMD Biosciences, 10394 Pacific Center Ct, San Diego, Calif. 92121, USA). The pDuet-1 vector containing the 57264 cassette is referred to herein as the plasmid pDuet:57264 (See FIG. 2B).

TABLE 1
Oligonucleotides used in this work

| Name | Sequence (orientation 5′-3′) | RE site |
|---|---|---|
| 57264F | AAAACCATGG CGGAAGCGGG CATTA | NcoI |
| NLF | AAAACCATGG TACCAAGCTT | NcoI |
| | AGGTGGTGGT GGTAGCG | |
| NLR | GCTTAAGGAT CCGCTACC | BamHI |
| NP40R | AAAAAAGCTT CCGCCTTCCC GCGG | HindIII |
| P40BAMHF | GATGGGACCC GACGCAACCA TTGAAA | — |
| P40BAMR | GCGTCGGGTC CCATCCGTCC CGGGT | — |
| CP40F3 | AAAAGGATCC GTGTTTCCAG CCACGCGA | BamHI |
| CP40R2 | AAAGAATTCA TTCACGCGGG | EcoRI |
| | TCACCAAATT CAT | |
| NPAF | AAAACCATGG TTACCCCGGC AGCGAATG | NcoI |
| NPAR | AAAAAAGCTT GCCTGGGCAT CATTCAG | HindIII |
| T7PROM | TAATACGACT CACTATAGGG | — |
| T7TERM | GCTAGTTATT GCTCAGCGG | — |
| M13RMG | TGTGGAATTG TGAGCGG | — |

The pDuet-1 plasmid is a duel promoter plasmid, and includes adjacent, duplicated T7 promoter regions flanked by a number of restriction sites. A simple digestion of the pDuet:57264 with the restriction enzymes XhoI and SalI, which have compatible cohesive ends, was performed, followed by recircularisation of the vector with T4 DNA ligase. The resulting plasmid is referred to herein as pDueT:57264SX (FIG. 2C).

A new linker region (NL) was designed, with new restriction sites incorporated, to allow rapid transfer of new domains into subsequent expression plasmids. The new linker was created by amplification from pDuet:57264 using the oligonucleotides NLF and NLR (see Table 1 and FIG. 2D). The NL PCR product was digested with the restriction enzymes NcoI and BamHI, and ligated into similarly digested pDuet:57264SX to replace the streptavidin domain and create the plasmid pDuetLNLCPA (see FIG. 2E).

The protein particle forming domain chosen for this exemplification was an N-terminal domain, designated P40, from a multi-domain beta-mannanase, ManA, from the bacterium Caldibacillus cellulovorans (Sunna et al, 2000. Appl. Environ. Microbiol. 66:664-670). The P40 domain encodes a protein that is homologous to known chitin binding domains. However, it was observed that the P40 domain, when expressed in E. coli, had no detectable carbohydrate binding affinity, and was expressed at high levels in the form of insoluble inclusion bodies.

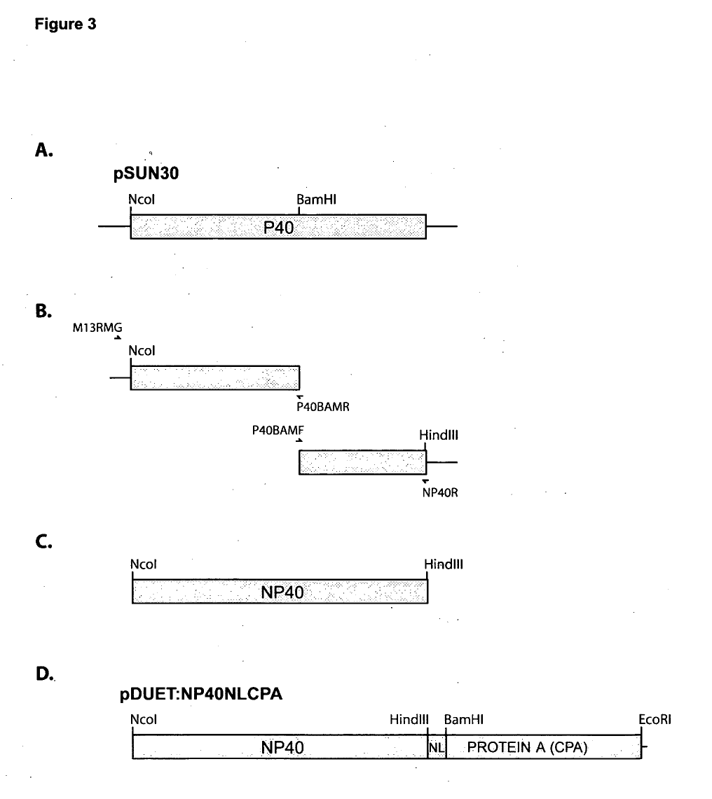

The region encoding the P40 domain was found to contain a single BamHI restriction site which we wished to remove to simplify downstream cloning procedures. A plasmid containing the P40 open reading frame, pSUN30 (see FIG. 3A), was used as template for PCR reactions. Three oligonucleotide primers, NP40R, P40BAMHF and P40BAMHR (see Table 1) were designed and synthesized for the simultaneous removal of the BamHI site and amplification of the P40 domain. The P40 domain was amplified by the PCR in two overlapping parts using the primer combinations M13RMG+P40BAMR and P40BAMF+NP40R (see FIG. 3B). The two parts were then recombined by overlap extension PCR to give a full length PCR product, designated NP40, now lacking an internal BamHI site (see FIG. 3C). The full length NP40 PCR product was then digested with the restriction enzymes NcoI and Hind III, and ligated into the same sites in the pDuet:NLCPA vector, to create the plasmid pDuet:NP40NLCPA (see FIG. 3D).

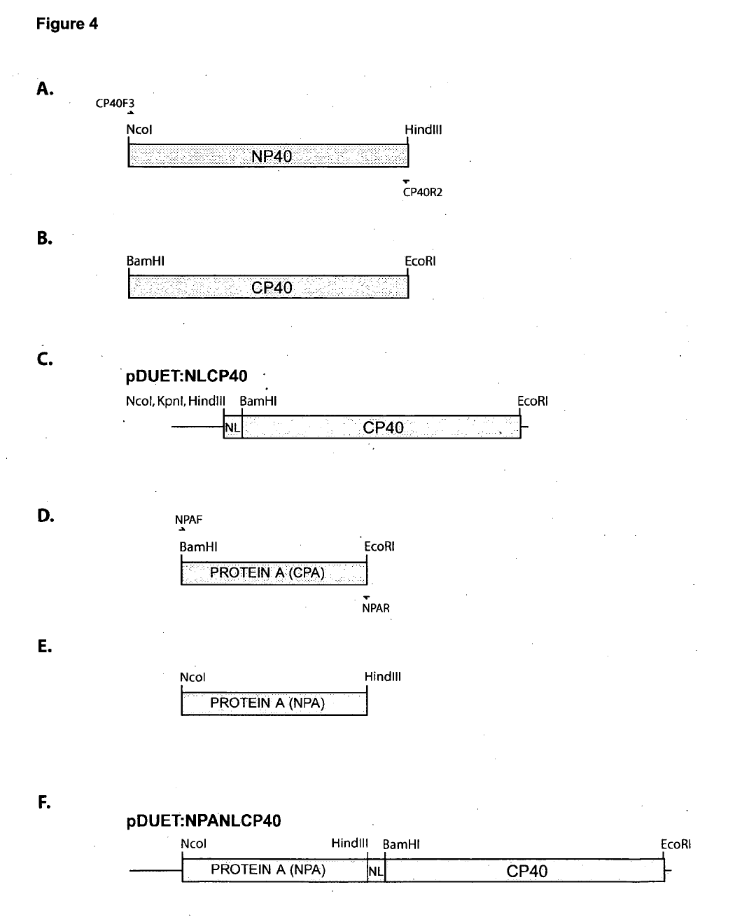

The oligonucleotide primers CP40F3 and CP40R2 (see Table 1) were used to PCR amplify the P40 domain from the plasmid pDuet:NP40NLCPA (see FIG. 4A). The CP40F3 and CP40R2 primers were designed to change the restriction sites flanking the P40 domain from NcoI+HindIII to BamHI and EcoRI (see FIG. 4B). The resulting PCR product, designated CP40, was then restriction digested with BamHI and EcoRI, then ligated into similarly digested pDuet:NLCPA to create the plasmid pDuet:NLCP40 (see FIG. 4C).

The oligonucleotide primers NPAF and NPAR (see Table 1) were used to PCR amplify the Protein A domain from the plasmid pDuet:NP40NLCPA (see FIG. 4D). The NPAF and NPAR primers were designed to change the restriction sites flanking the Protein A domain from BamHI and EcoRI to NcoI+Hind III (see FIG. 4E). The resulting PCR product, designated NPA, was then restriction digested with NcoI+HindIII, then ligated into similarly digested pDuet:NLCP40 to create the plasmid pDuet:NPANLCP40 (see FIG. 4F).

### Expression of Chimeric Proteins in the Form of Self Assembling Protein Particles

The E. coli strain BL21-tuner (Novagen) was used as an expression host for the plasmids pDuet:NP40NLCPA and pDuet:NPANLCP40. Single recombinant bacterial colonies were picked and seeded directly into 50 ml of the autoinduction medium Magicmedia (Invitrogen Corporation) supplemented with 100 μg ml⁻¹ ampicllin Cultures were grown overnight for approximately 24 hours at 37° C. Post-induction, cells were then harvested by centrifugation at 2300×g for 10 minutes, the supernatant discarded, and the cell pellet resuspended in 3 ml sterile deionised water by vigorous mixing, to give a final volume of 4 ml.

Purification of Insoluble Protein Fraction from E. coli Cells

The 4 ml of resuspended Recombinant E. coli BL21-tuner cells were lysed by passing twice through a French pressure cell. The 4 ml of cell lysate was then combined with 16 ml BPER [Phosphate] solution (Pierce Biotechnology Inc, Rockford, Ill. 61105, USA) and 4 mg of lysozyme, and mixed for 15 minutes. The insoluble fraction was then pelleted by centrifugation at 12000×g, for 30 min at 4° C. The supernatant was then decanted and the pellet washed by resuspension in 15 ml of a 1 in 10 solution of BPER diluted in sterile deionised water. The insoluble fraction was then pelleted again by centrifugation at 12000×g, for 30 min at 4° C., then washed again, before finally being resuspended in 10 ml of sterile deionised water.

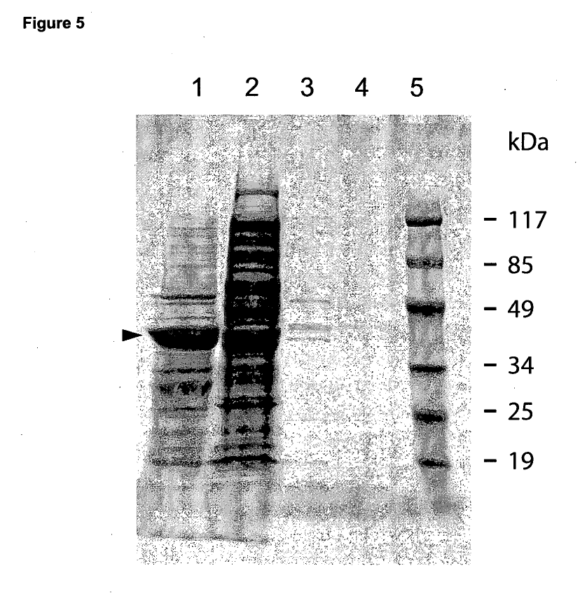

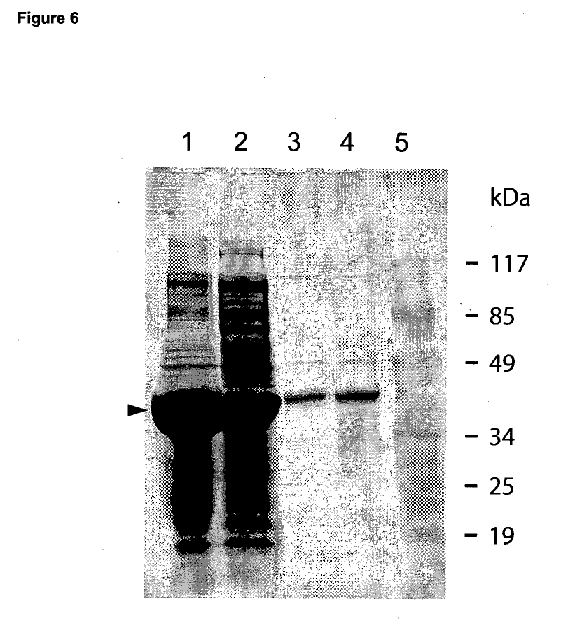

The insoluble fractions were analysed by SDS-PAGE to determine the size and relative amounts of recombinant protein within the insoluble, soluble and wash fractions as depicted in FIG. 5 and FIG. 6.

### Example 2

### Analysis of Protein Particles

This example visualises protein particles purified from recombinant E. coli, and shows the functional binding of the NP40NLCPA Protein A domain (CPA) to fluorescently labelled mouse specific goat antibody. Functional binding was visualized by two methods a) direct binding of fluorescently labelled anti-mouse goat antibody to CPA, or b) primary labelling of the CPA with an anti-EHV1 mouse polyclonal antibody, followed by secondary labelling of the mouse antibody with the fluorescently labelled anti-mouse goat antibody.

The methods used for labelling and visualisation were as follows:
I. Purified protein particles (40 μl) of NP40NLCPA and a control inclusion body particle without a Protein A domain, called NP40NHSABP, were pelleted by centrifugation in a microcentrifuge for 1 minute at 14000 rpm. A 20 μl aliquot of Sigma ProtG Sepharose 4B fast flow beads (Sigma-Aldrich) was also pelleted using the same conditions and used as a positive control.
II. Supernatants were removed and the pellets resuspended by pipetting in 100 μl wash buffer (1× phosphate buffer saline (PBS), 10% Fetal bovine serum [FBS]). Steps 1 & 2 were repeated.
III. Resuspended particles/beads were then divided into separate tubes in 20 μl aliquots.
IV. Molecular probes Alexafluor 488 Goat anti-rat antibody and Alexafluor 546 Goat anti-mouse antibody (Invitrogen Corporation, Carlsbad, Calif. 92130, USA) were diluted 1 in 1000 in wash buffer.
V. Individual aliquots of each particle/bead were then mixed with 100 μl of either diluted antibody, then incubated at room temperature for 1 hour with gentle mixing.
VI. Aliquots were then pelleted and washed twice again by centrifugation as above.
VII. Samples were finally resuspended in 100 μl PBS.
VIII. A total of 2 μl of each labelled particle/bead was placed on a slide, covered and visualised by DIC and confocal laser scanning microscopy.
IX. Aliquots of NP40NLCPA and Prot G sepharose beads from step III were also incubated with a 1:50 dilution of anti-EHV1 polyclonal antibody (mouse) for 1 hour, washed twice, then labelled as per steps 4-6 with Alexafluor 546 Goat anti-mouse antibody, before being resuspended directly in 10 μl mountant, placed on a slide, covered, and visualised by DIC and confocal laser scanning microscopy with the appropriate wavelength lasers and filters.

An assay to determine binding specificity of NP40NLCPA inclusion bodies towards goat IgG antibodies was carried out. Each sample was analysed by two views: flattened confocal Z-stacked image and DIC image. NP40NLCPA particles were labelled with Alexafluor488 goat anti-mouse IgG antibody, NP40NLCPA particles were labelled with Alexafluor546 goat anti-mouse IgG antibody. The labelled NP40NLCPA particles were compared with ProtG sepharose 4b fast flow beads labelled with Alexafluor 546 goat anti-mouse IgG antibody and ProtG sepharose 4b fast flow beads labelled with Alexafluor 488 goat anti-mouse IgG antibody.

An assay to determine binding specificity of NP40NLCPA protein particles towards mouse IgG antibodies was carried out. Each sample was analysed by two views: confocal flat image and DIC image. NP40NLCPA was labelled with a 1:50 dilution of EHV1 polyclonal primary antibody (mouse), then labelled with Alexafluor546 secondary goat anti-mouse IgG antibody.

The NP40NLCPA protein particles were observed to bind specifically to fluorescently labelled goat anti-mouse IgG, and also to bind specifically to mouse IgG detected by addition of the secondary, fluorescently labelled goat anti-mouse IgG.

### Example 3

### Use of Functional Protein Particles for Separation of Immunoglobulins from Serum

Various amounts of NPANLCP40 and NP40NLCPA protein particles as well as protein particles not containing protein A domain (negative control) were spun down in microcentrifuge tubes (see Table 2)

The particles were then washed with the following solutions:

3×1 ml 20 mM Tris, 1M NaCl pH 7.8

1×1 ml 50 mM glycine pH 1.9

1×1 ml 1M Tris pH 8.0

2×1 ml TBS

Each tube was then incubated with 1 ml of human serum diluted 1+4 in TBS. Incubation time: 45 minutes at room temperature.

The particles were then washed with the following solutions:

2×1 ml TBS

1×20 mM Tris, 1M NaCl pH 7.8

1×20 mM Tris pH 7.8

The particles were then eluted with 2×100 μl 50 mM glycine pH 1.9 and the elutes were pooled. Protein concentration in the elutes were measured in a 1+9 dilution with TBS.

TABLE 2

| | | OD280 | Concentration | Total | mg IgG/ |
|---|---|---|---|---|---|
| | | (dil. | of IgG in | amount of | ml of |
| Particles | Amount | 1 + 9) | elute | IgG | particles |
| NPANLCP40 | ~50 μl | 0.133 | 0.89 mg/ml | 0.178 mg | 3.6 |
| NP40NLCPA | ~5 μl | 1.540 | 10.3 mg/ml | 2.06 mg | 412 |
| Negative | ~25 μl | 0.017 | — | — | — |
| control | | | | | |

### SDS-PAGE Analysis of Elutes:

100 μl of each elute was neutralised with 10 μl 1M Tris pH 8

### Running of Gel:

Gel: NuPage 4-12% Bis-Tris gel (Invitrogen)

Running buffer: MES buffer

Run time: 40 min@200V

Marker: Mark12 Unstained standard (Invitrogen)

Loading buffer: 1 ml NuPage LDS+5% β-mercaptoethanol

Staining: Coomassie Blue

All samples (apart from markers): 12 μl sample+4 μl loading buffer, followed by boiling for 5 min and then spun down in microcentrifuge tube.

### Loading of Gel:

M: Marker; 5 μl

S: Human serum (diluted 1+4); 5 μl

1: Elute of NPANLCP40; 5 μl

2: Elute of NP40NLCPA (diluted 1+1); 5 μl

3: Elute of negative control; 5 μl

4: Elute of NPANLCP40; 10 μl

5: Elute of NP40NLCPA (diluted 1+1); 10 μl

6: Elute of negative control; 10 μl

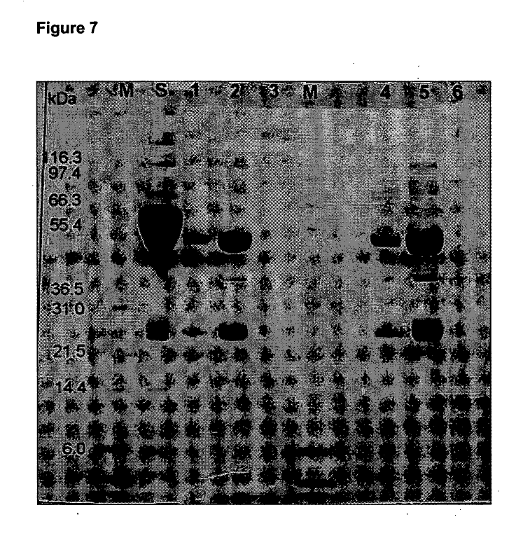

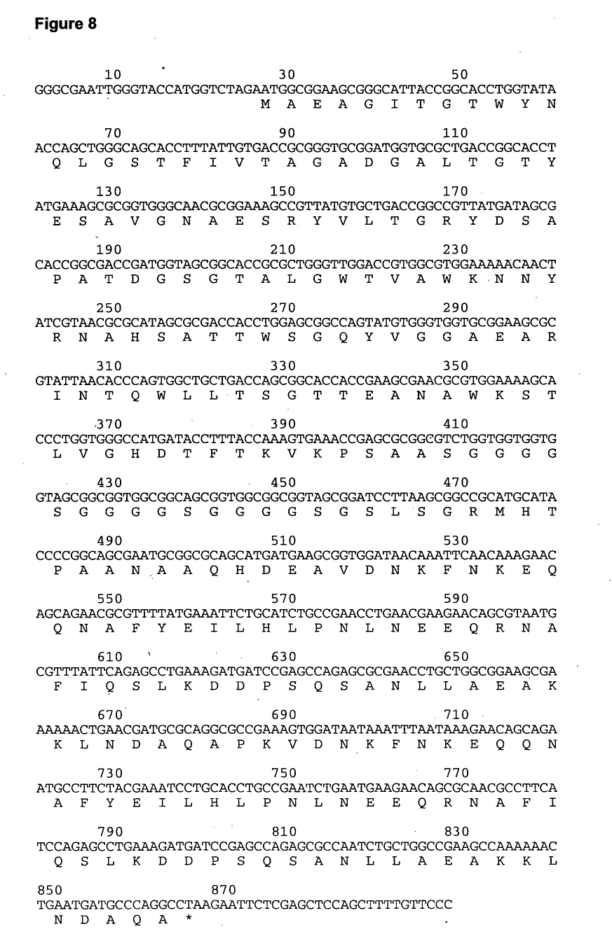

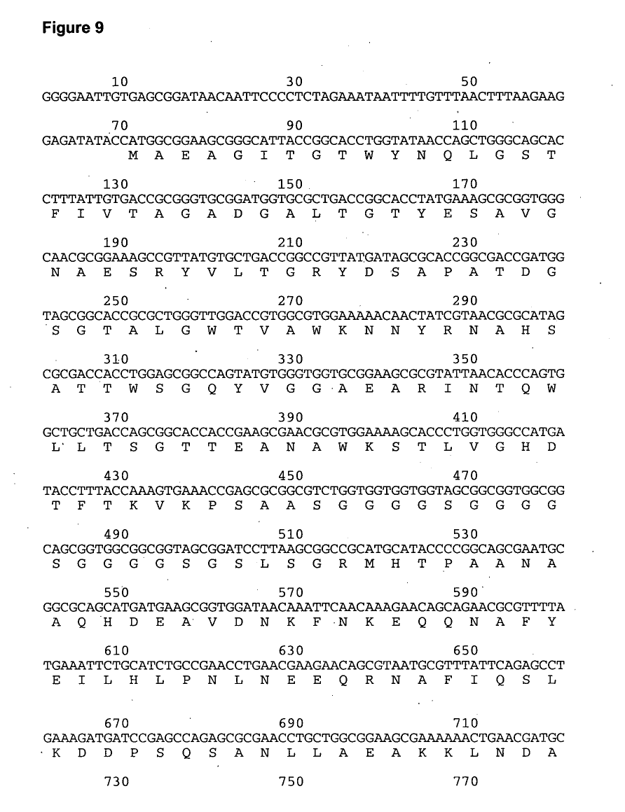

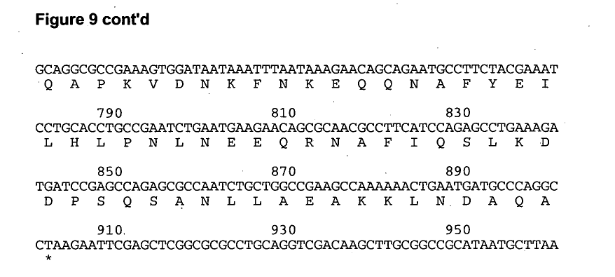

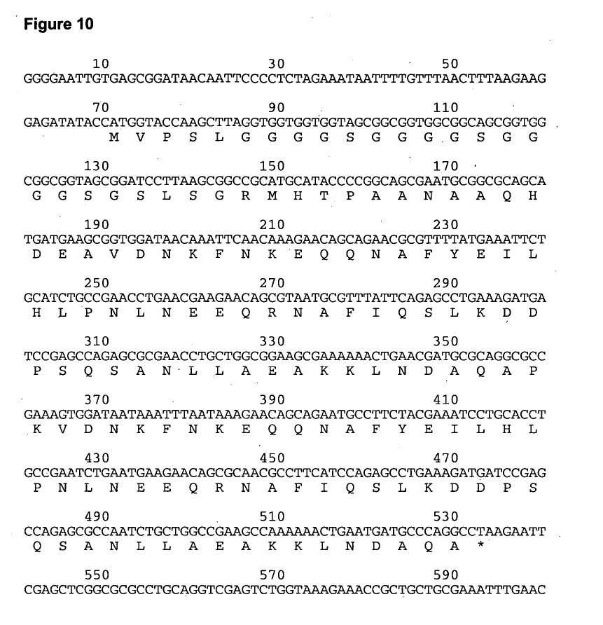

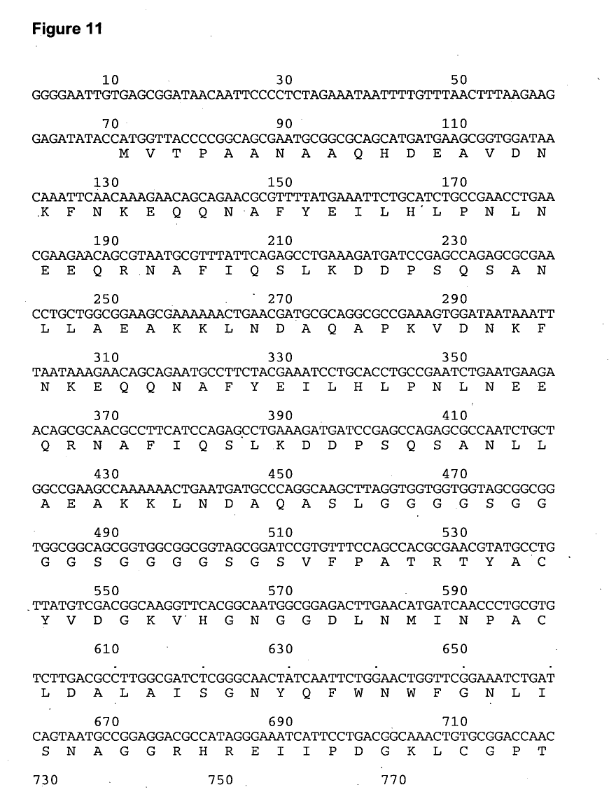

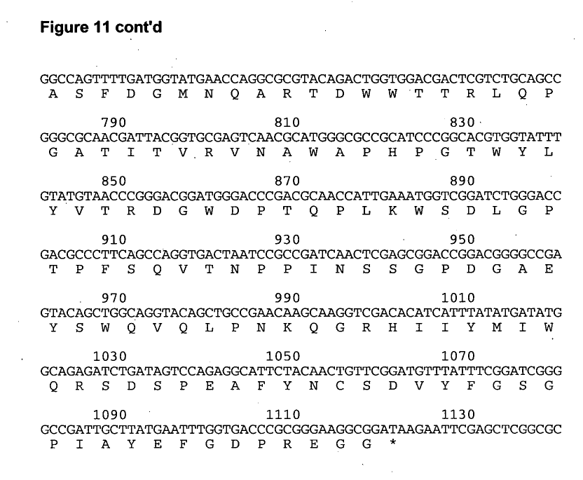

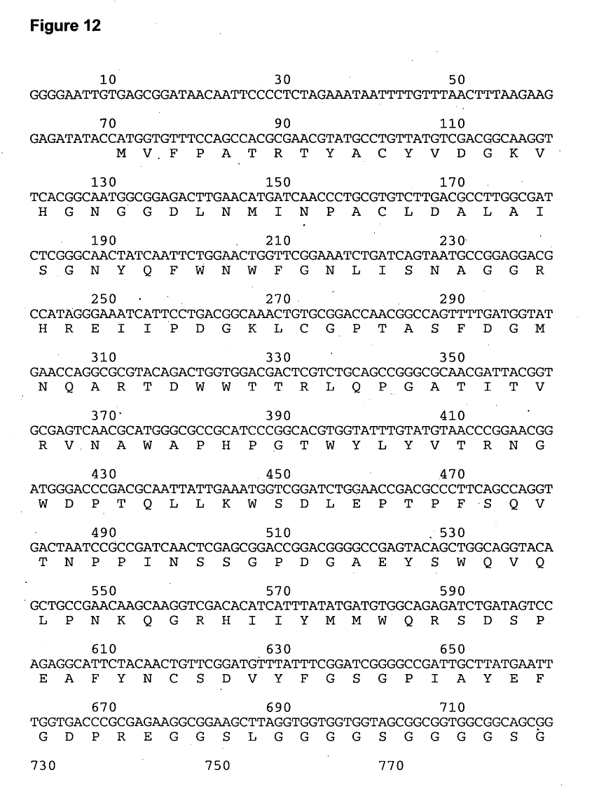

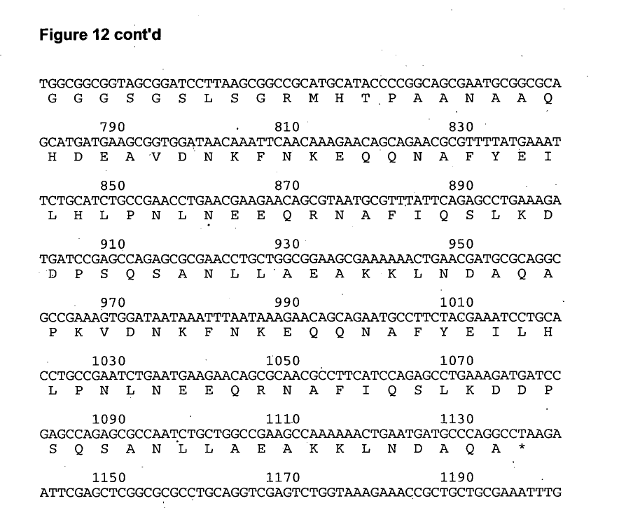

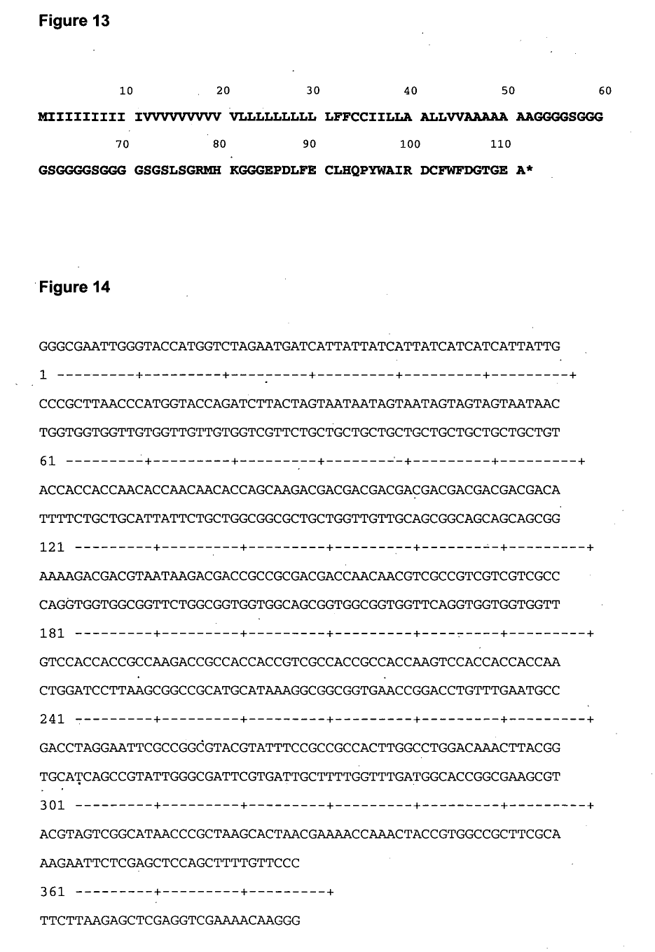

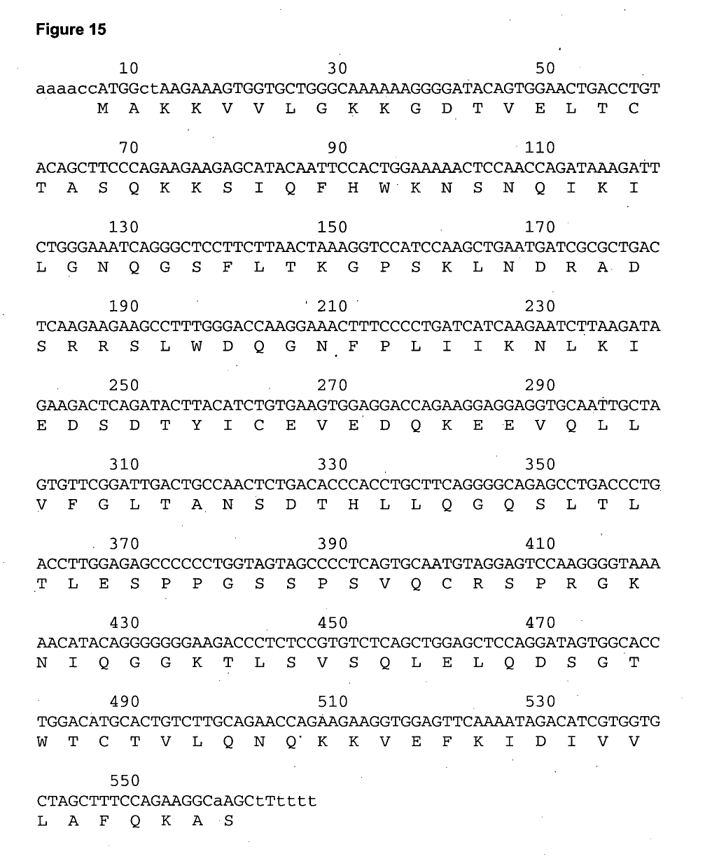

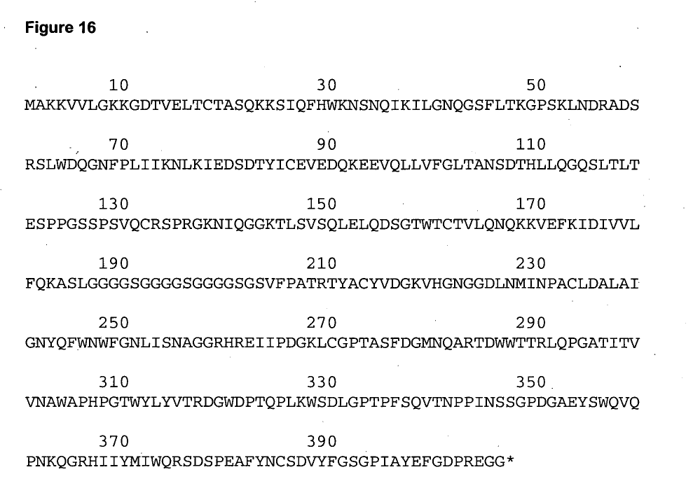

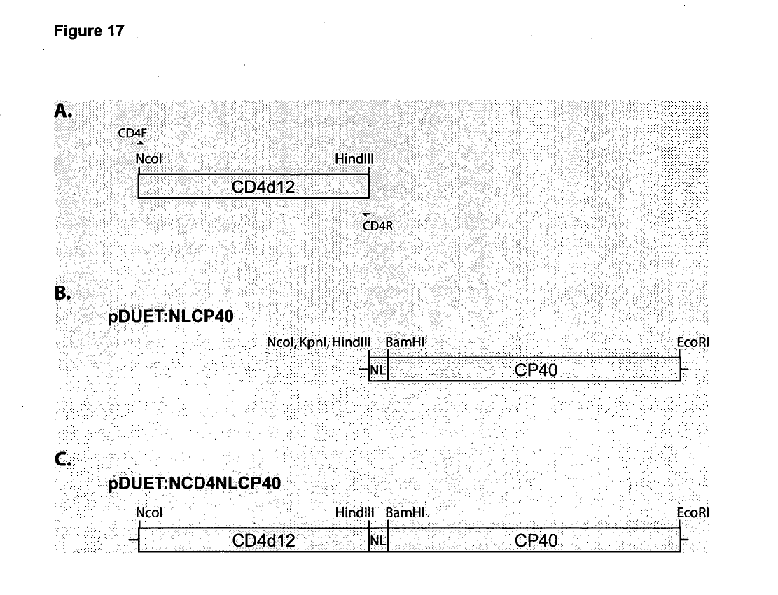

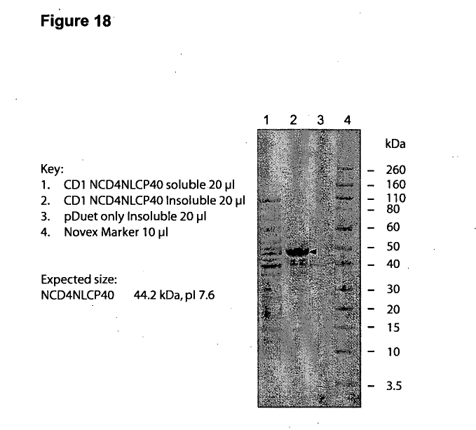

The presence of purified/enriched heavy and light chains of immunoglobulins on the SDS-PAGE image shown on FIG. 7 demonstrate that immunoglobulins were purified/enriched from the human serum using the protein particle affinity matrix according to the present invention. The results furthermore indicate, that the affinity particles prepared according to the present invention are capable of purifying immunoglobulins from serum to a degree of purity comparable to that obtained by using affinity beads prepared by traditional methods.

### Example 4

### Synthetic Particle Forming Domain

In addition to the use of proteins or protein domains that are know to form inclusion bodies or aggregates when expressed in appropriate organisms, synthetic protein domains can also be created which can serve as the particle forming part of the fusion proteins. One method of making a synthetic particle forming protein is to create a DNA construct encoding a protein domain with a large proportion of hydrophobic amino acids. When such a domain is expressed in an appropriate host microorganism, this protein domain will aggregate and thus form protein particles.

An example of a fusion protein with a synthetic particle forming domain is shown in FIG. 13 and an example of a nucleotide sequence encoding such a fusion protein is shown in FIG. 14. It will be appreciated that particle forming protein domains can be constructed by means other than by creating hydrophobic domains, and the example given is therefore not meant to restrict particle formation to aggregation of hydrophobic protein domains.

### Example 5

### Creation of a PNP with a CD4 Domains

This example shows creation of a N-terminal fusion of human CD4 domains 1 and 2 (CD4d12) to a PNP-forming domain.

The N-terminal domains of CD4 (CD4d12) in Streptomyces lividans can be expressed as a secreted protein. The protein has been determined to be correctly folded and biologically active by immunoprecipitation assays using HIV envelope glycoprotein GP120. CD4d12 is functional and can bind to GP120 when secreted from Lactobacillus jensenii.

The following method describes the creation of a recombinant N-terminal fusion of CD4d12 to the PNP-forming P40 domain.

The region encoding the N-terminal domains of human CD4 (CD4d12) was amplified from the plasmid pT4luc using the oligonucleotide primers CD4F and CD4R. The pT4luc plasmid contains the complete human CD4 cDNA (Maerz et al, J. Virol. 75:6635-6644, 2001). The CD4F and CD4R primers were designed to incorporate the restriction sites NcoI and HindIII respectively, to allow digestion and directional ligation of the PCR product into similarly digested plasmid. The CD4d12 PCR product was digested with NcoI and HindIII, gel-purified, then ligated into the plasmid pDuet:NLCP40, to create the plasmid pDuet:NCD4NLCP40. The ligation mix was transformed into competent E. coli BL21 Tuner cells and plated onto LB-agar plates containing 100 μg ml ampicillin. Recombinant colonies were picked, cultured, and plasmid DNA prepared. Plasmid DNA was sequenced, and the DNA sequence analysed and confirmed error free.

The confirmed sequence of the PCR product is shown in FIG. 15. The predicted translated gene product from pDuet:NCD4NLCP40 is shown in FIG. 16. An overview of the gene construction is shown in FIG. 17. SDS-Page analysis of the expressed construct is shown in FIG. 18. The SDS PAGE analysis clearly shows that insoluble protein particles were produced when the fusion protein construct was expressed in E. coli.

The oligonucleotide primer sequences used are as follows:

| (SEQ ID NO: 16) | |
|---|---|
| CD4F | 5′-AAAACCATGGCTAAGAAAGTGGTGCTGGGCA |
| (SEQ ID NO: 17) | |
| CD4R | 5′-AAAAAAGCTTGCCTTCTGGAAAGCTAGCA |

### CD4 Expression and Purification

To prepare recombinant protein, 5 ml overnight cultures of each recombinant isolate were grown in LB medium containing 100 μg/ml ampicillin then used to seed 100 ml of fresh medium containing antibiotic. A control strain containing the plasmid pDuet1 was selected and the culture was then grown at 37° C. with shaking until the cell density reached an absorbance at 590 nm of approximately 1.5. IPTG was then added at a final concentration of 0.05 mM, and the cells grown a further 3 hours. Cells were harvested by centrifugation, then lysed by two passages through a French pressure cell.

Then insoluble fraction of the lysate was harvested by centrifugation at 18,000 rpm for 30 min. The pellet was the fully resuspended in BPER (Pierce) and inclusion bodies purified as per the BPER manufacturers recommendations. The purified inclusion bodies were examined by SDS-PAGE electrophoresis as shown in FIG. 18.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A protein particle comprising:
chimeric protein having an aggregating part capable of forming or aggregating into a substantially insoluble protein particle when expressed by a cell; and
a functional part capable of binding to, or being bound by, a target compound.

2. The protein particle according to claim 1 wherein the aggregating part is a synthetic peptide, naturally occurring peptide or mutant peptide capable of forming aggregates.

3. The protein particle according to claim 2 wherein the aggregating part is P40.

4. The protein particle according to any one of claims 1 to 3 wherein the functional part comprises protein A, protein G, protein L, an antibody binding domain, a single chain antibody, avidin, streptavidin, an enzyme, an inhibitor, an antigenic determinant, an epitope, a binding site, a lectin, a cellulose binding protein, a polyhistidine, an oligohistidine, a receptor, a hormone, a signalling molecule, a polypeptide with specific or group specific binding capabilities, or a combination thereof.

5. The protein particle according to claim 4 wherein the functional part comprises protein A.

6. The protein particle according to any one of claims 1 to 5 comprising two or more functional parts capable of binding to, or being bound by, a two or more target compounds.

7. A nucleic acid molecule encoding a protein particle according to any one of claims 1 to 6 comprising:
a nucleic acid molecule encoding a chimeric protein having an aggregating protein part and a functional protein part capable of binding to, or being bound by, a target compound;
wherein when the nucleic acid molecule is expressed in a cell, a protein which can form a particle capable of binding to, or being bound by, a target compound is formed.

8. A method of forming a protein particle capable of binding to, or being bound by,
a target compound comprising:
providing a nucleic acid molecule according to claim 7 to a cell;
allowing the cell to express the nucleic acid molecule to form an insoluble protein particle; and
recovering the insoluble protein particle.

9. The method according to claim 8 wherein the nucleic acid molecule is provided to the cell in a construct selected from vector, plasmid, virus, or any other suitable expression means.

10. An affinity matrix for binding or separating at least one target component from a mixture comprising a protein particle according to any one of claims 1 to 6.

11. The affinity matrix according to claim 10 comprising a plurality of protein particles.

12. The affinity matrix according to claim 11 comprising a protein particle containing one or more different functional parts capable of binding one or more different target components.

13. A method for binding at least one target component from a mixture comprising:
providing a sample containing a target component to an affinity matrix according to any one of claims 10 to 12; and
allowing a target component in the sample to bind to the functional part of the matrix.

14. A method for recovering at least one target component from a mixture comprising:
providing a sample containing a target component to an affinity matrix according to any one of claims 10 to 12;
allowing a target component in the sample to bind to the functional part of the matrix, and
recovering the target component from the matrix.

15. The method according to claim 13 or 14 wherein the target component includes a protein, a peptide, a polypeptide, an immunoglobulin, biotin, an inhibitor, a co-factor, a substrate, an enzyme, a receptor, a monosaccharide, an oligosaccharide, a polysaccharide, a glycoprotein, a lipid, a nucleic acid, a cell or fragment thereof, a cell extract, an organelle, a microorganism, virus, a biological extract, a hormone, a serum protein, a milk protein, a milk-derived product, blood, serum, plasma, a fermentation product, a macromolecule or any other molecule or any combination or fraction thereof.

16. Use of the affinity matrix according to any one of claims 10 to 12 to separate or enrich at least one target component.

17. A kit for affinity separation comprising:
an affinity matrix according to any one of claims 10 to 12; and
diluent and/or eluent for carrying out an affinity separation using the matrix.

18. The kit according to claim 17 further including instructions to carry out an affinity separation.
